(19) United States
(12) Patent Application Publication
(10) Pub. No.: US 2024/0299371 A1
(43) Pub. Date: Sep. 12, 2024

(54) BIARYL AMIDE AND HETEROARYL AMIDES FOR TREATMENT OF CANDIDA ALBICANS INFECTION

(71) Applicant: Board of Regents, The University of Texas System, Austin, TX (US)

(72) Inventors: STANTON MCHARDY, San Antonio, TX (US); JOSE LOPEZ-RIBOT, San Antonio, TX (US); MATTHEW VALDEZ, San Antonio, TX (US); JESUS ROMO, San Antonio, TX (US); STEPHEN SAVILLE, San Antonio, TX (US)

(21) Appl. No.: 18/282,083

(22) PCT Filed: Mar. 15, 2022
(86) PCT No.: PCT/US2022/020439

§ 371 Date: Sep. 14, 2023

Related U.S. Application Data
(60) Provisional application No. 63/161,124, filed on Mar. 15, 2021.

(51) Int. Cl. A61K 31/444; A61K 31/337; A61K 31/4427; C07D 305/06; C07D 405/12; C07D 405/14

(52) U.S. Cl. CPC A61K 31/444; A61K 31/337; A61K 31/4427; C07D 305/06; C07D 405/12; C07D 405/14

(57) ABSTRACT

Embodiments described herein address the problem of treating or ameliorating fungal infections and biofilm formation, and in particular Candida infection. Certain aspects are directed to anti-fungal compounds and compositions.

This application is an International application claiming priority to U.S. Provisional Application Ser. No. 63/161,124 filed Mar. 15, 2021, which is incorporated herein by reference in its entirety.

### STATEMENT REGARDING FEDERALLY FUNDED RESEARCH

This invention was made with government support under 5R01AI119554 awarded by the National Institutes of Health. The government has certain rights in the invention.

### BACKGROUND

Biofilms possess unique developmental characteristics that are in stark contrast to the characteristics of free-floating cells, and biofilms are much more difficult to treat chemotherapeutically. A biofilm is a structured community of microorganisms encapsulated within a self-developed polymeric matrix and adherent to a living or inert surface. Biofilms are also often characterized by surface attachment, structural heterogeneity, genetic diversity, complex community interactions, and an extracellular matrix of polymeric substances. Formation of a biofilm begins with the attachment of free-floating microorganisms to a surface. These first colonists adhere to the surface initially through weak, reversible van der Waals forces and can anchor themselves using cell adhesion structures such as pili. The first colonists facilitate the arrival of other cells by providing more diverse adhesion sites and beginning to build the matrix that holds the biofilm together. Once colonization has begun, the biofilm grows through a combination of cell division and recruitment.

Biofilms can form in or on medical equipment, such as catheters, and are a major source of hospital infections. A primary difficulty with biofilms is that they are more difficult to reduce or eliminate as compared to free-floating organisms. This is due to the formation of a protective layer of a biofilm as well as adaptations that the individual organisms undergo when they form biofilms. Microorganisms living in a biofilm can have significantly different properties as compared to free-floating microorganisms. The dense and protected environment of the film allows them to cooperate and interact in various ways. One benefit of this environment is increased resistance to detergents and antibiotics, as the dense extracellular matrix and the outer layer of cells protect the interior of the community.

The yeast Candida albicans is a commensal of human mucosal surfaces and is also an opportunistic pathogen. Candida albicans causes a wide variety of diseases including oral thrush and disseminated candidiasis. Systemic fungal infections have emerged as important causes of morbidity and mortality in immunocompromised patients. In addition, hospital-related infections in patients not previously considered at risk have become a cause of major health concern. Candida albicans is also the primary fungus colonizing medical devices. Infections involving medical devices are notoriously difficult to eliminate and generally necessitate removal of the device. Candida albicans colonizes the surfaces of catheters, prostheses, implants, and epithelia forming biofilms that are extremely resistant to antifungal drugs. Mature Candida albicans biofilms show a complex three-dimensional architecture with extensive spatial heterogeneity, and consist of a dense network of yeast, hyphae and pseudohyphae encased within a matrix of exopolymeric material. An estimated 50% of hospital infections are caused by biofilms formed on medical devices (Kojic et al., Cin Microbiol Rev 2004, 17:255-267).

The azoles, such as fluconazole, are one major class of effective antifungals; they act through inhibition of ergosterol biosynthesis. Candida albicans azole resistance has been known for some time in the clinic, and several resistance mechanisms are well characterized. (Pranab et al., Infect Immun, 2003, 71: 4333-4340). LaFleur et al. reported that biofilms formed by Candida albicans exhibited a strikingly biphasic killing pattern in response to microbicidal agents amphotericin B and chlorhexidine indicating that a subpopulation of highly tolerant cells, termed persisters, existed. (LaFleur et al., Antimicrob Agents Chemother, 2006 50:3839-46.)

Thus, there remains a need for additional compositions and methods for treating fungal infections such as Candida infections.

### SUMMARY

In addressing the problem of fungal infections and biofilm formation, and in particular Candida infection, a solution has been discovered in the form of new molecules that are effective anti-fungals. This invention is directed toward compounds of Formula I or a pharmaceutically acceptable salt thereof, and methods of their use.

Certain embodiments are directed to compounds of Formula I:

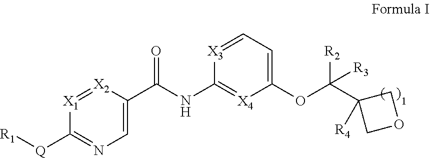

wherein:

In certain aspects, R₁ is hydrogen (H); C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; C₃, C₄, C₅, C₆, or C₇ cycloalkyl. In certain aspects the alkyl or cycloalkyl is substituted. R₁ can be substituted with 1, 2, or 3 halogens; a C₁, C₂, or C₃ alkyl; a C₃, C₄, C₅, C₆, or C₇ cycloalkyl; a heterocycle; aryl; or heteroaryl. In particular aspects, the alkyl or the cycloalkyl is substituted with a methyl or a substituted methyl.

In certain aspects, R₂, R₃, and R₄ are independently selected from hydrogen (H); C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; C₃, C₄, C₅, C₆, or C₇ cycloalkyl. In certain aspects the alkyl or cycloalkyl is substituted. When R₂, R₃, and/or R₄ is not hydrogen, R₂, R₃, and/or R₄, respectively, can be substituted with 1, 2, or 3 halogens; a C₁, C₂, or C₃ alkyl; a C₃, C₄, C₅, C₆, or C₇ cycloalkyl; a heterocycle; aryl; or heteroaryl. In particular aspects, the alkyl or the cycloalkyl is substituted with a methyl or a substituted methyl.

In certain aspects, n can be 1, 2, or 3.

In certain aspects Q is oxygen or a sigma bond;

In certain aspects, X₁, X₂, X₃, and X₄ are independently CH, nitrogen (N), or a carbon substituted with a halogen, a nitrogen, or an R₁ group as defined above.

According to another aspect, pharmaceutical compositions are provided that include a compound of Formula I as described above. The pharmaceutical compositions can include a pharmaceutically acceptable carrier and any compound embraced by Formula I described above.

Certain embodiments are directed to methods of treating fungal infections by administering one or more compounds described above. In some embodiments, the fungal infection is caused by a yeast of the Candida genus. In one embodiment, the yeast is of the Candida albicans species. In other embodiments, the Candida yeast may be of the Candida dubliniensis, Candida parapsilosis, Candida tropicalis, Candida kerfyr, Candida guilliermondii, Candida inconspicua, Candida famata, Candida glabrata, Candida krusei, Candida lusitaniae, or other Candida species.

In the methods described for treating fungal infections, a subject in need of such treatment is administered one of the compounds described above in an amount effective to treat the fungal infection.

The subject can be a human, a non-human primate, or other mammal. In certain embodiments, the subject is a human. In some embodiments, the subject is immunocompromised. In certain aspects the subject is hospitalized or in need of hospitalization. A subject can be administered a compound of Formula I prophylactically prior to entering a facility or location that may harbor Candida or other fungi. In other embodiments the subject is a non-human vertebrate selected from the group consisting of a dog, cat, horse, cow, pig, turkey, goat, fish, monkey, chicken, rat, mouse, and sheep.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

Throughout this application, the term “about” is used to indicate that a value includes the inherent variation of error for the device, the method being employed to determine the value, or the variation that exists among the study subjects.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

### DESCRIPTION

Fungi are eukaryotic organisms that are classified generally based on morphological features, modes of reproduction, and culture characteristics. Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, Candida albicans, Cryptococcus neoformans, and various Aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi. Diseases associated with fungal infection include candidiasis. The compounds described herein are useful for treating diseases associated with fungal infection either alone or in combination with existing anti-fungal therapies.

Candidiasis is a fungal infection caused by a member of the genus candida. The disease can be in the form of allergic, cutaneous, mucocutaneous, or systemic candidiasis. Nystatin is used for the treatment of the cutaneous, mucocutaneous, and allergic diseases. Amphotericin B is useful for treating this systemic disease. Other drugs useful for the treatment include 5-fluorocytosine, fluconazole, itraconazole and voriconazole.

The pathogenicity of C. albicans can be attributed to its ability to survive and thrive in multiple microenvironments within a host, including multiple organs, the mucosa, and the bloodstream, as well as to virulence factors that aid in the adherence and invasion of multiple cell types (Brown et al., 1999. Trends Microbiol. 7: 333-338; Mitchell, 1998, Curr. Opin. Microbiol. 1: 687-692; Odds, 1994, J. Am. Acad. Dermatol. 31: S2-S5). C. albicans exists within immunocompetent individuals in a commensal relationship on mucosal linings of the oral cavity, esophagus, vagina, gastrointestinal tract, etc. Oropharyngeal, esophageal, vulvovaginal, and cutaneous candidiasis leads to significant morbidity. Lethality is often associated with systemic infections in immunocompromised patients. Systemic infections arise from colonization of mucosal surfaces by adherence to epithelial cells, followed by the penetration of epithelial and endothelial cell barriers and dissemination throughout the body (Filler et al., 1995, Infect. Immun. 63: 976-983).

### I. ANTI-FUNGAL COMPOUNDS

Certain embodiments are directed to bi-heteroaryl amide compounds as inhibitors of Candida albicans biofilms and treatments for Candida albicans-related infections.

Certain embodiments are directed to compounds of Formula I or pharmaceutically acceptable salts thereof:

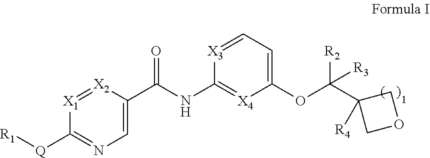

wherein:

In certain aspects, R₁ is hydrogen (H); C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; C₃, C₄, C₅, C₆, or C₇ cycloalkyl. In certain aspects the alkyl or cycloalkyl is substituted. R₁ can be substituted with 1, 2, or 3 halogens; a C₁, C₂, or C₃ alkyl; a C₃, C₄, C₅, C₆, or C₇ cycloalkyl; a heterocycle; aryl; or heteroaryl. In particular aspects, the alkyl or the cycloalkyl is substituted with a methyl or a substituted methyl.

In certain aspects, R₂, R₃, and R₄ are independently selected from hydrogen (H); C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; C₃, C₄, C₅, C₆, or C₇ cycloalkyl. In certain aspects the alkyl or cycloalkyl is substituted. When R₂, R₃, and/or R₄ is not hydrogen, R₂, R₃, and/or R₄ can be substituted with 1, 2, or 3 halogens; a C₁, C₂, or C₃ alkyl; a C₃, C₄, C₅, C₆, or C₇ cycloalkyl; a heterocycle; aryl; or heteroaryl. In particular aspects, the alkyl or the cycloalkyl is substituted with a methyl or a substituted methyl.

In certain aspects, n can be 1, 2, or 3.

In certain aspects Q is oxygen or a sigma bond;

In certain aspects, X₁, X₂, X₃, and X₄ are independently CH, nitrogen (N), or a carbon substituted with a halogen, a nitrogen, or an R₁ group as defined above.

The compounds of Formula I may be prepared by the methods described below, together with synthetic methods known in the art of organic chemistry, or modifications and derivatizations that are familiar to those of ordinary skill in the art. Preferred methods include, but are not limited to, those described below. During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Synthesis. Compounds of Formula I or their pharmaceutically acceptable salts, can be prepared according to the following reaction Schemes below. Isolation and purification of the products is accomplished by standard procedures, which are known to a chemist of ordinary skill.

The following schemes are examples of the processes for making compounds of Formula I. It is to be understood, however, that the invention, as fully described herein and as recited in the claims, is not intended to be limited by the details of the following examples. Unless otherwise noted, X₁, X₂, X₃, X₄, Q, n, R₁ R2, R₃ and R₄ are defined as above.

The preparation of the biheteroaryl amide compounds of general Formula I are described below in Scheme 1.

Treatment of an appropriately substituted nicotinic acid 1 with oxalyl chloride in THE produces the corresponding acid chloride, which is immediately reacted with the general aniline 2 to give the desired amide 3. Alternative conditions for this reaction sequence could involve acid chloride generation with alternative reagents such as SOCl₂, and the like, or direct amide coupling reactions between the acid 1 and the aniline 2 could be carried out under standard methods, such as DCC, HATU, and the like. Hydrogenation of the resulting benzyl protected biaryl amide 3 with pallidum on carbon in alcoholic solvents such as MeOH or EtOH, in the presence of a hydrogen gas atmosphere at pressures from 1 atmosphere to 100 psi, produces the desired intermediate hydroxy-pyridine (X═CH) derivative 4. Finally, alkylation of 4 with various substituted halo-methyl oxetanes of general structure 5 provides compounds of general Formula I.

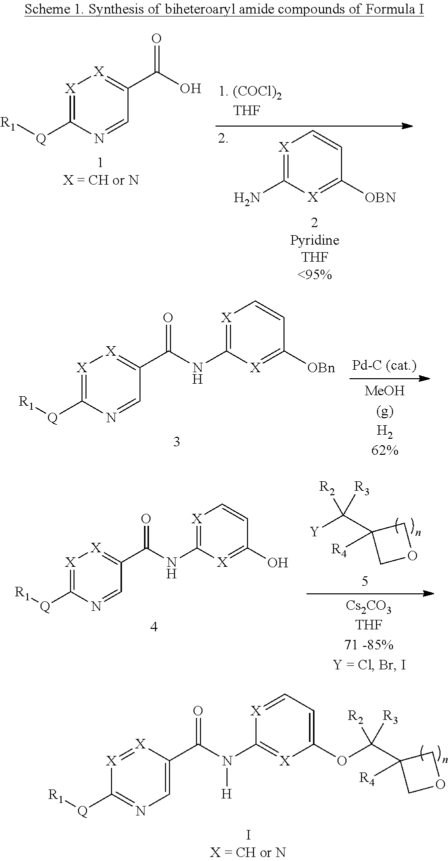

Finally, pharmaceutically acceptable salts of compounds of Formula I may be prepared by one or more of three methods:
(i) by reacting the compound of Formula I with the desired acid;
(ii) by removing an acid-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

Pharmaceutically acceptable salts of the compounds of Formula I include the acid addition salts thereof. All three reactions are typically carried out in solution. The resulting salt may precipitate out and be collected by filtration or may be recovered by evaporation of the solvent. The degree of ionization in the resulting salt may vary from completely ionized to almost non-ionized.

Suitable acid addition salts are formed from acids which form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts. For a review on suitable salts, see Handbook of Pharmaceutical Salts: Properties, Selection, and Use by Stahl and Wermuth (Wiley-VCH, 2002).

Included within the scope of the present invention are all stereoisomers, geometric isomers, atropisomers and tautomeric forms of the compounds of Formula I, including compounds exhibiting more than one type of isomerism, and mixtures of one or more thereof.

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature. The present invention also includes all pharmaceutically acceptable pro-drug derivatives of Formula I or Formula II, as well as all biotinylated analogs.

Compounds of Formula I can be assessed for activity against Candida albicans biofilms and infections by these non-limiting methods. Most frequently the methodology uses the 96-well microtiter plate technique for the formation and susceptibility testing as originally developed and described by the Lopez-Ribot group (Standardized method for in vitro antifungal susceptibility testing of Candida albicans biofilms. G Ramage, K V Walle, B L Wickes, J L López-Ribot—Antimicrobial agents and chemotherapy, 2001; A simple and reproducible 96-well plate-based method for the formation of fungal biofilms and its application to antifungal susceptibility testing. C G Pierce, P Uppuluri, A R Tristan, F L Wormley Jr—Nature protocols, 2008). Briefly, a standardized cell suspension of C. albicans is added to the wells of microtiter plates in the absence or presence of serial dilutions of the investigational compound in order to assess the biofilm-inhibitory activity of the compound under investigation. The assay is coupled to an XTT-reduction colorimetric assay that measures metabolic activity of cells within the biofilms, and these colorimetric readings can be used to assess % inhibition and to determine the potency of the investigational compound (typically by calculating its IC₅₀). Likewise, biofilms of C. albicans can be formed using the same method, and after biofilm formation serial dilutions of the compound under investigation can be added to the wells of the microtiter plate, thereby determining its activity against preformed (mature) biofilms.

Chemical Definitions—Various chemical definitions related to such compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

As used herein, the term “water soluble” means that the compound dissolves in water at least to the extent of 0.010 mole/liter or is classified as soluble according to literature precedence.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “alkyl,” by itself or as part of another substituent, means, unless otherwise stated, a linear (i.e., unbranched) or branched carbon chain, which may be fully saturated, monounsaturated or polyunsaturated. An unsaturated alkyl group is one having one or more double bonds or triple bonds. Unsaturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂ Br, —CH₂ OH, —CH₂ OCH₃, —CH₂ OCH₂ CF₃, —CH₂OC(O)CH₃, —CH₂ NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂ Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Examples of optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g., “C₁₋₄ alkylsulfonyl”)

The term “monosaccharide” refers to a cyclized monomer unit based on a compound having a chemical structure H(CHOH)ₙC(═O)(CHOH)ₘH wherein n+m is 4 or 5. Thus, monosaccharides include, but are not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

It is contemplated that any embodiment discussed in this specification can be implemented with respect to any method or composition of the invention, and vice versa. Furthermore, compositions of the invention can be used to achieve methods of the invention.

### II. METHODS OF TREATING

Certain embodiments are directed to methods of treating fungal infections by administering one or more compounds described above. In the methods for treating fungal infections provided herein, the compounds described above are administered in effective amounts. An effective amount means that amount necessary to delay the onset of, inhibit the progression of, halt altogether the onset or progression of, or diagnose the particular infection being treated. When administered to a subject, effective amounts will depend on the particular condition being treated, the severity of the condition, individual patient parameters including age, physical condition, size and weight, concurrent treatment, frequency of treatment, and the mode of administration. These factors are well known to those of ordinary skill in the art and can be addressed with no more than routine experimentation. It is preferred generally that a maximum dose be used, that is, the highest safe dose according to some medical judgment.

In another embodiment, the compounds of the present invention may also be used in combination with other anti-fungal agents such as azoles (e.g., fluconazole), echinocandins (e.g., caspofungin) and polyenes (e.g., Amphotericin B). These techniques use the same 96-well microtiter plate model of C. albicans biofilm formation in which biofilms are exposed to multiple combinations of the investigational compound and the established antifungal prepared using a checkerboard pattern.

The compounds of this invention can be administered for the prevention and/or treatment of candidiasis (in its different manifestations, i.e. oral candidiasis, vaginal candidiasis, systemic and invasive candidiasis, etc.) via either the oral, parenteral (such as subcutaneous, intraveneous, intramuscular, intrasternal and infusion techniques), rectal, intranasal or topical routes to mammals, as well as intra-catheter (which allows for the use of suprapharmacological concentrations within the catheter lumen). In general, these compounds are most desirably administered to humans in doses ranging from about 1 mg to about 2000 mg per day, although variations will necessarily occur depending upon the weight and condition of the subject being treated and the particular route of administration chosen. However, a dosage level that is in the range of from about 0.1, 0.5, 1, 2, 3, 4, 5, 6, 7, 8, 9, 10 mg to about 11, 12, 13, 14, 15, 16, 17, 18, 19, 20 mg per kg of body weight per day (including all values and ranges there between) can be employed. Nevertheless, variations may still occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects provided that such higher dose levels are first divided into several small doses for administration throughout the day.

As used herein, the term treat, treated, or treating when used with respect to an disorder such as an infectious disease refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

Generally, daily doses of active compounds will be from about 0.01 milligrams/kg per day to 1000 milligrams/kg per day. It is expected that oral doses in the range of 0.5 to 50 milligrams/kg, or even 1 to 10 milligrams/kg per day, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. It is expected that intravenous and other parenteral forms of administration will yield the desired results in the range of 0.1 to 10 milligrams/kg per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

When administered, the formulations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic. Also, pharmaceutically acceptable salts can be prepared as alkaline metal or alkaline earth salts, such as sodium, ammonium, magnesium, potassium or calcium salts of the carboxylic acid group.

Suitable buffering agents include: acetic acid and salts thereof (1 to 2% W/V); citric acid and salts thereof (1 to 3% W/V); boric acid and salts thereof (0.5 to 2.5% W/V); and phosphoric acid and salts thereof (0.8 to 2% W/V). Suitable preservatives include benzalkonium chloride (0.003 to 0.03% W/V); chlorobutanol (0.3 to 0.9% W/V); parabens (0.01 to 0.25% W/V) and thimerosal (0.004 to 0.02% W/V).

A variety of administration routes are available. The particular mode selected will depend, of course, upon the particular compound or combination of drugs selected, the severity of the condition or disorder being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, transdermal, sublingual or intramuscular, infusion, intraparenteral, intravenous, intramuscular, intracavity, as a feed additive, as an aerosol, aurally (e.g., via eardrops), intranasal, inhalation, or subcutaneous. Direct injection could also be preferred for local delivery to the site of infection. Oral administration may be preferred for prophylactic treatment because of the convenience of the subject (patient) as well as the dosing schedule.

For oral administration, the compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the compounds. The compound of the invention is delivered to the lungs of a mammal while inhaling and traverses across the lung epithelial lining to the blood stream. Examples of reports of inhaled molecules include Adjei et al., 1990, Pharmaceutical Research, 7: 565-569; Adjei et al., 1990, International Journal of Pharmaceutics, 63: 135-144 (leuprolide acetate); Braquet et al., 1989, Journal of Cardiovascular Pharmacology, 13(suppl. 5): 143-146 (endothelin-1); Hubbard et al., 1989, Annals of Internal Medicine, Vol. III, pp. 206-212 (al-antitrypsin); Smith et al., 1989, J. Clin. Invest. 84: 1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140: 3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

The compositions may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

The compounds of the invention may optionally be delivered with other antifungal agents in the form of antifungal cocktails, or individually, yet close enough in time to have a synergistic effect on the treatment of the infection. An antifungal cocktail is a mixture of any one of the above-described compounds with another antifungal drug which may or may not be a compound of the invention. The use of such cocktails in pharmaceutical preparations is routine. In this embodiment, a common administration vehicle (e.g., tablet, implants, injectable solution, injectable liposome solution, etc.) could contain both the compound of the invention and the other antifungal agent(s).

Anti-fungal agents are useful for the treatment and prevention of infective fungi. Some of these treatments are described above. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Antifungal agents include Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Cancidas (Caspofungin Acetate), Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; and Zinoconazole Hydrochloride.

### III. EXAMPLES

The following examples as well as any figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All operations were carried out at room or ambient temperature, that is, in the range of 18 to 25° C.; evaporation of solvent was carried out using a rotary evaporator under reduced pressure with a bath of up to 50° C.; reactions were monitored by thin layer chromatography (tlc) and reaction times are given for illustration only. Purity of all isolated compounds were assured by at least one of the following techniques: tlc (Merck silica gel 60 F-254 precoated plates), high performance liquid chromatography (HPLC), mass spectrometry and/or nuclear magnetic resonance (NMR). Yields are given for illustrative purposes only. Flash column chromatography was carried out using Merck silica gel 60 or utilizing a Biotage Isolera One purification systems with UV detection and automatic fraction collections. Low-resolution mass spectral data (EI) were obtained on an Agilent 1290 HPLC with Agilent 6150 MS. Liquid Chromatography data was collected on an Agilent 1260 Infinity HPLC w/variable wavelength UV detection and auto sampler. NMR data was determined at 400 MHz (Agilent DD2 400 MHz Nuclear Magnetic Resonance Spectrometer) using deuterated chloroform (99.8% D), methanol (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br=broad, etc.

The following abbreviations are used: THF—tetrahydrofuran, DCM or CH₂Cl₂—dichloromethane; NaHCO₃:—sodium bicarbonate; HCl—hydrogen chloride; MgSO₄—magnesium sulfate; Na₂SO₄—sodium sulfate; DME—dimethoxyethane; n-BuLi—n-butyllithium; DMF—dimethylformamide; DMSO—dimethylsulfoxide; Et₂O—diethyl ether; MeOH—methanol; EtOAc—ethyl acetate

### Example I

### Representative Procedure for the Formation of General Amide Structure

Part A: To a flame-dried and nitrogen purged flask equipped with a stir bar, added a solution of the appropriately substituted carboxylic acid (1 equiv) in THE (30 mL, 0.33 M) followed by the dropwise of oxalyl chloride (1.5 equiv) and 3 drops of DMF which resulted in the evolution of gas. After approximately one to two hours, an aliquot was removed from the reaction and diluted with methanol. Reaction progressed was measured by the disappearance of the carboxylic acid starting material and the formation of the resulting methyl ester as determined by TLC or NMR. Upon consumption of the starting material, the reaction was concentrated down under reduced pressure and placed on a high-vacuum pump overnight.

Part B: The appropriately substituted amino-phenol (1 equiv) was added to a round bottom flask equipped with a stir bar and added THE (30 mL, 0.33 M) followed by pyridine (2 equiv). An addition funnel was placed onto the flask and a solution of the freshly prepared benzoyl chloride (from the carboxylic acid starting material) in THE (0.5 M) was placed in the addition funnel. Over the course of 30 minutes to an hour, the benzoyl chloride solution was added to the flask. Upon consumption of the benzoyl chloride intermediate as determined by TLC or LCMS the reaction was quenched with a solution of aqueous 1 M HCl and the resulting solid was filtered and collected via vacuum filtration to give 88 as a solid. Substrates bearing additional nitrogen atoms were quenched with water in place of aqueous 1 M HCl and extracted with a 3:1 chloroform/IPA mixture three times, dried with sodium sulfate, and concentrated under reduced pressure to give yellow/white solids.

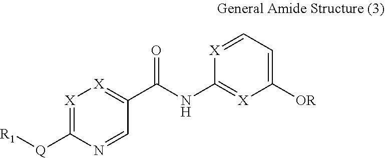

### Example 2

### Representative Procedure for the Formation of General Formula I Through the Alkylation of Phenols

To a 40 mL scintillation vial equipped with stir bar, added phenol (1 equiv), Cs₂CO₃ (2.2 equiv), and DMF (0.5 M). Allowed the mixture to stir for 15 minutes then added the appropriate alkyl bromide 5 (1.1 equiv). The reaction was allowed to stir for 12-24 hours. Upon completion as determined by TLC or LC-MS, the reaction was quenched with water and diluted with ethyl acetate. The solution was transferred to a separatory funnel and the aqueous layer was extracted with EtOAc. The aqueous layer was extracted with EtOAc three times, dried over Na₂SO₄, and concentrated under reduced pressure. Purification crude product was carried out via by flash chromatography to afford the desired compounds of general structure 89. Lead compound 8 and compounds 95-139 were prepared using the general method described above.

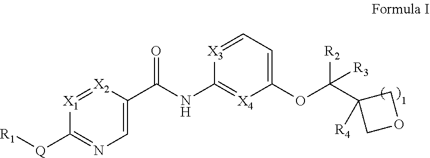

### Example 3

### Representative Examples and Characterization of Compounds of Formula I

### 6-methoxy-N-(3-(oxetan-3-ylmethoxy)phenyl)nicotinamide

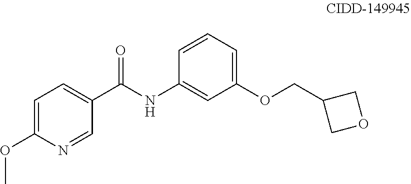 [SMILES: COc1ccc(C(=O)Nc2cccc(OCC3COC3)c2)cn1]

¹H NMR (400 MHz, DMSO-d₆) δ 8.74 (d, J=2.0 Hz, 1H), 8.19 (dd, J=8.7, 2.5 Hz, 1H), 7.45 (t, J=2.1 Hz, 1H), 7.29 (d, J=8.8 Hz, 1H), 7.21 (t, J=8.1 Hz, 1H), 6.91 (d, J=8.7 Hz, 1H), 6.67 (dd, J=8.1, 1.6 Hz, 1H), 4.68 (dd, J=7.9, 6.1 Hz, 2H), 4.40 (t, J=6.0 Hz, 2H), 4.15 (d, J=6.7 Hz, 2H), 3.90 (s, 2H). ¹³C NMR (100 MHz, CDCl₃) δ 166.2, 163.9, 159.4, 146.4, 138.9, 137.7, 129.8, 123.9, 112.6, 111.1, 106.6, 74.3, 69.1, 53.9, 34.5. ESI-MS m/z [M+H]⁺:315.2

### 6-methoxy-N-(3-((3-methyloxetan-3-yl)methoxy)phenyl)nicotinamide

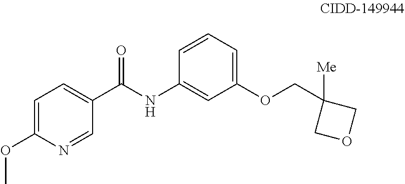 [SMILES: COc1ccc(C(=O)Nc2cccc(OCC3(C)COC3)c2)cn1]

¹H NMR (400 MHz, DMSO-d₆) δ 10.18 (s, 1H), 8.74 (d, J=2.1 Hz, 1H), 8.19 (dd, J=8.7, 2.5 Hz, 1H), 7.48 (t, J=2.1 Hz, 1H), 7.29 (d, J=8.8 Hz, 1H), 7.22 (t, J=8.1 Hz, 1H), 6.92 (d, J=8.7 Hz, 1H), 6.70 (dd, J=8.1, 1.6 Hz, 1H), 4.46 (d, J=5.8 Hz, 2H), 4.28 (d, J=5.8 Hz, 2H), 4.00 (s, 2H), 3.90 (s, 3H), 1.34 (s, 3H). ESI-MS m/z [M+H]⁺:329.3.

### 4-methoxy-N-(3-(oxetan-3-ylmethoxy)phenyl)benzamide

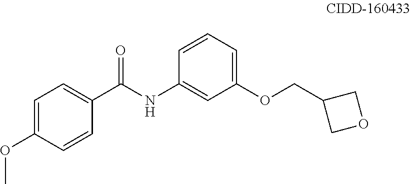 [SMILES: COc1ccc(C(=O)Nc2cccc(OCC3COC3)c2)cc1]

¹H NMR (400 MHz, CDCl₃) δ 7.84 (d, J=8.0 Hz, 2H), 7.77 (s, 1H), 7.54 (s, 1H), 7.28-7.22 (m, 2H), 7.03 (d, J=7.9 Hz, 1H), 6.98 (d, J=8.0 Hz, 2H), 6.70 (d, J=8.3 Hz, 1H), 4.88 (t, J=6.7 Hz, 2H), 4.58 (t, J=5.8 Hz, 2H), 4.23 (d, J=6.7 Hz, 2H), 3.88 (s, J=9.3 Hz, 3H), 3.45 (dt, J=13.5, 6.7 Hz, 1H). ¹³C NMR (100 MHz, CDCl₃) δ 165.1, 162.5, 159.3, 139.3, 129.7, 128.8, 127.0, 113.9, 112.3, 110.8, 106.2, 74.2, 69.1, 5.4, 34.5. ESI-MS m/z [M+H]⁺: 314.2

### 4-methoxy-N-(3-((3-methyloxetan-3-yl)methoxy)phenyl)benzamide

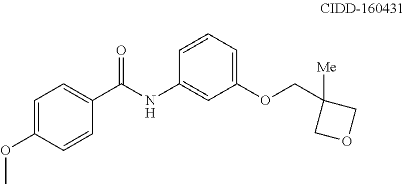 [SMILES: COc1ccc(C(=O)Nc2cccc(OCC3(C)COC3)c2)cc1]

¹H NMR (400 MHz, DMSO) δ 10.00 (s, 1H), 7.92 (d, J=8.7 Hz, 2H), 7.51 (s, 1H), 7.32 (d, J=8.2 Hz, 1H), 7.21 (t, J=8.1 Hz, 1H), 7.03 (d, J=8.7 Hz, 2H), 6.68 (d, J=6.5 Hz, 1H), 4.47 (d, J=5.7 Hz, 2H), 4.28 (d, J=5.7 Hz, 2H), 4.00 (s, 2H), 3.81 (s, 3H), 1.34 (s, 3H). ¹³C NMR (100 MHz, DMSO) δ 165.3, 162.3, 159.3, 140.9, 129.9, 129.7, 127.3, 114.0, 113.1, 109.9, 107.18, 79.0, 72.8, 55.8, 21.4. ESI-MS m/z [M+H]⁺: 328.2

### 4-methoxy-N-(3-((3-ethyloxetan-3-yl)methoxy)phenyl)benzamide

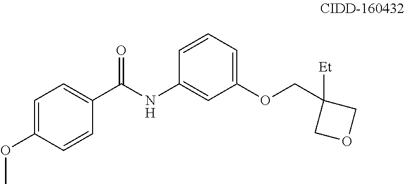 [SMILES: CCC1(COc2cccc(NC(=O)c3ccc(OC)cc3)c2)COC1]

¹H NMR (400 MHz, CDCl₃) δ 7.92 (s, 1H), 7.82 (d, J=8.8 Hz, 2H), 7.54 (t, J=2.1 Hz, 1H), 7.22 (t, J=8.5 Hz, 1H), 7.03 (dd, J=8.0, 1.1 Hz, 1H), 6.94 (d, J=8.8 Hz, 2H), 6.70 (dd, J=8.2, 1.8 Hz, 1H), 4.55 (d, J=6.0 Hz, 2H), 4.45 (d, J=6.0 Hz, 2H), 4.07 (s, J=6.2 Hz, 2H), 3.84 (s, 3H), 1.85 (q, J=7.5 Hz, 2H), 0.91 (t, J=7.5 Hz, 3H). ¹³C NMR (100 MHz, CDCl₃) δ 165.2, 162.4, 159.6, 139.4, 129.6, 128.8, 127.0, 113.9, 112.3, 110.7, 106.4, 78.1, 70.2, 55.4, 43.1, 26.7, 8.1. ESI-MS m/z [M+H]⁺: 342.2.

### 4-methoxy-N-(3-((3-ethyloxetan-3-yl)methoxy)phenyl)benzamide

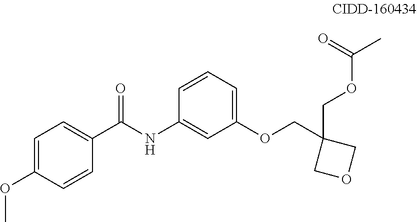 [SMILES: COc1ccc(C(=O)Nc2cccc(OCC3(COC(C)=O)COC3)c2)cc1]

¹H NMR (400 MHz, DMSO) δ 10.02 (s, 1H), 7.95-7.89 (m, 2H), 7.51 (t, J=2.1 Hz, 1H), 7.33 (d, J=9.1 Hz, 1H), 7.22 (t, J=8.1 Hz, 1H), 7.03 (d, J=8.9 Hz, 2H), 6.69 (dd, J=8.1, 1.8 Hz, 1H), 4.47 (d, J=6.2 Hz, 2H), 4.43 (d, J=6.2 Hz, 2H), 4.33 (s, 2H), 4.15 (s, 2H), 3.81 (s, 3H), 2.02 (s, 3H). ¹³C NMR (100 MHz, DMSO) δ 170.9, 165.3, 162.3, 159.0, 140.9, 129.9, 129.8, 127.3, 114.0, 113.3, 109.9, 107.2, 74.8, 69.0, 64.9, 55.8, 42.6, 21.0. ESI-MS m/z [M+H]⁺: 386.2, [M+Na]⁺: 408.2

### 6-methoxy-N-(4-(oxetan-3-ylmethoxy)pyridin-2-yl)nicotinamide

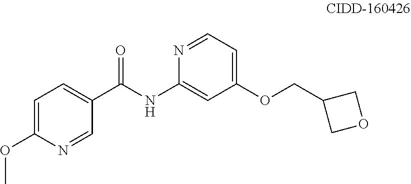 [SMILES: COc1ccc(C(=O)Nc2cc(OCC3COC3)ccn2)cn1]

¹H NMR (400 MHz, CDCl₃) δ 8.85 (s, 1H), 8.75 (d, J=2.2 Hz, 1H), 8.09 (dd, J=8.7, 2.5 Hz, 1H), 8.03 (d, J=5.8 Hz, 1H), 8.00 (d, J=2.2 Hz, 1H), 6.81 (d, J=8.7 Hz, 1H), 6.61 (dd, J=5.8, 2.3 Hz, 1H), 4.87 (dd, J=7.7, 6.4 Hz, 2H), 4.55 (t, J=6.1 Hz, 2H), 4.31 (d, J=6.7 Hz, 2H), 3.99 (s, 3H), 3.51-3.40 (m, 1H). ESI-MS m/z [M+H]⁺: 316.2.

### 6-methoxy-N-(4-((3-methyloxetan-3-yl)methoxy)pyridin-2-yl)nicotinamide

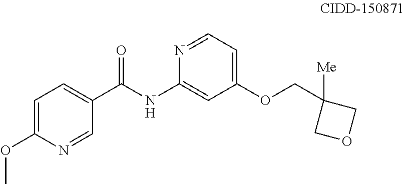 [SMILES: COc1ccc(C(=O)Nc2cc(OCC3(C)COC3)ccn2)cn1]

¹H NMR (400 MHz, CDCl₃) δ 8.75 (d, J=2.4 Hz, 1H), 8.59 (s, 1H), 8.16-8.04 (m, 2H), 8.01 (d, J=2.2 Hz, 1H), 6.82 (d, J=8.7 Hz, 1H), 6.65 (dd, J=5.8, 2.3 Hz, 1H), 4.61 (d, J=6.0 Hz, 2H), 4.45 (d, J=6.0 Hz, 2H), 4.14 (s, 2H), 4.00 (s, 3H), 1.44 (s, 3H). ¹³C NMR (100 MHz, DMSO) δ 165.3, 162.3, 159.3, 140.9, 129.9, 129.7, 127.3, 114.0, 113.1, 109.9, 107.18, 79.0, 72.8, 55.8, 21.4. ESI-MS m/z [M+H]⁺: 330.2

### 6-methoxy-N-(4-((3-ethyloxetan-3-yl)methoxy)pyridin-2-yl)nicotinamide

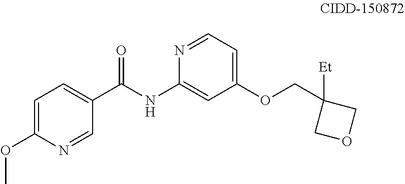 [SMILES: CCC1(COc2ccnc(NC(=O)c3ccc(OC)nc3)c2)COC1]

¹H NMR (400 MHz, DMSO) δ 10.83 (s, 1H), 8.85 (d, J=2.1 Hz, 1H), 8.29 (dd, J=8.7, 2.2 Hz, 1H), 8.22 (d, J=5.7 Hz, 1H), 7.86 (s, 1H), 6.93 (d, J=8.7 Hz, 1H), 6.85 (dd, J=5.7, 1.9 Hz, 1H), 4.47 (d, J=6.0 Hz, 2H), 4.36 (d, J=5.9 Hz, 2H), 4.21 (s, 2H), 3.94 (s, 3H), 1.80 (q, J=7.4 Hz, 2H), 0.91 (t, J=7.4 Hz, 3H). ESI-MS m/z [M+H]⁺: 344.2.

### N-(4-((3-(hydroxymethyl)oxetan-3-yl)methoxy)pyridin-2-yl)-6-methoxynicotinamide

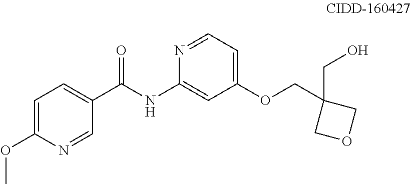 [SMILES: COc1ccc(C(=O)Nc2cc(OCC3(CO)COC3)ccn2)cn1]

¹H NMR (400 MHz, DMSO) δ 10.80 (s, 1H), 8.82 (d, J=2.4 Hz, 1H), 8.25 (dd, J=8.7, 2.5 Hz, 1H), 8.18 (d, J=5.7 Hz, 1H), 7.83 (d, J=2.2 Hz, 1H), 6.89 (d, J=8.7 Hz, 1H), 6.80 (dd, J=5.7, 2.3 Hz, 1H), 5.01 (t, J=4.7 Hz, 1H), 4.42 (d, J=6.0 Hz, 2H), 4.37 (d, J=6.0 Hz, 2H), 4.21 (s, 2H), 3.90 (s, 3H), 3.69 (d, J=4.3 Hz, 2H). ¹³C NMR (100 MHz, DMSO) δ 166.5, 165.9, 164.7, 154.0, 149.3, 148.3, 139.3, 123.9, 110.5, 107.5, 100.8, 74.9, 69.0, 62.6, 54.2, 44.3. ESI-MS m/z [M+H]⁺: 346.2.

### N-(3-(oxetan-3-ylmethoxy)phenyl)nicotinamide

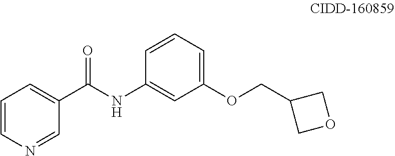 [SMILES: O=C(Nc1cccc(OCC2COC2)c1)c1cccnc1]

¹H NMR (400 MHz, DMSO) δ 10.40 (s, 1H), 9.10 (d, J=1.6 Hz, 1H), 8.76 (dd, J=4.8, 1.6 Hz, 1H), 8.32-8.24 (m, 1H), 7.57 (ddd, J=8.0, 4.8, 0.7 Hz, 1H), 7.51 (t, J=2.1 Hz, 1H), 7.35 (d, J=8.8 Hz, 1H), 7.28 (d, J=8.1 Hz, 1H), 6.76-6.72 (m, 1H), 4.72 (dd, J=7.9, 6.0 Hz, 2H), 4.44 (t, J=6.0 Hz, 2H), 4.20 (d, J=6.7 Hz, 2H), 3.46-3.34 (m, 1H). ¹³C NMR (100 MHz, DMSO) δ δ 164.5, 159.1, 152.5, 149.0, 140.4, 135.8, 130.9, 129.9, 123.9, 113.2, 110.4, 107.1, 73.5, 69.3, 34.3. ESI-MS m/z [M+H]⁺: 285.2.

### N-(3-((3-methyloxetan-3-yl)methoxy)phenyl)nicotinamide

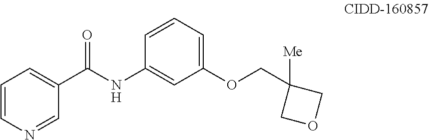 [SMILES: CC1(COc2cccc(NC(=O)c3cccnc3)c2)COC1]

¹H NMR (400 MHz, DMSO) δ 10.42 (s, 1H), 9.11 (d, J=1.7 Hz, 1H), 8.76 (dd, J=4.8, 1.5 Hz, 1H), 8.32-8.27 (m, 1H), 7.59-7.53 (m, 2H), 7.36 (d, J=8.9 Hz, 1H), 7.28 (t, J=8.1 Hz, 1H), 6.76 (dd, J=8.1, 1.7 Hz, 1H), 4.50 (d, J=5.8 Hz, 2H), 4.32 (d, J=5.8 Hz, 2H), 4.04 (s, 2H), 1.38 (s, 3H). ¹³C NMR (100 MHz, DMSO) δ 165.3, 162.3, 159.3, 140.9, 129.9, 129.7, 127.3, 114.0, 113.1, 109.9, 107.18, 79.0, 72.8, 55.8, 21.4. ESI-MS m/z [M+H]⁺: 299.2.

### N-(3-((3-ethyloxetan-3-yl)methoxy)phenyl)benzamide

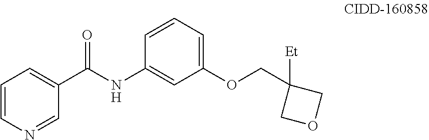 [SMILES: CCC1(COc2cccc(NC(=O)c3cccnc3)c2)COC1]

¹H NMR (400 MHz, DMSO) δ 10.39 (s, 1H), 9.10 (d, J=1.6 Hz, 1H), 8.76 (dd, J=4.8, 1.6 Hz, 1H), 8.32-8.26 (m, 1H), 7.57 (ddd, J=8.0, 4.9, 0.8 Hz, 1H), 7.54 (t, J=2.1 Hz, 1H), 7.35 (d, J=8.8 Hz, 1H), 7.28 (t, J=8.1 Hz, 1H), 6.80-6.75 (m, 1H), 4.46 (d, J=5.9 Hz, 2H), 4.35 (d, J=5.9 Hz, 2H), 4.09 (s, 2H), 1.80 (q, J=7.5 Hz, 2H), 0.91 (t, J=7.5 Hz, 3H). ESI-MS m/z [M+H]⁺: 313.3.

### N-(3-((3-(hydroxymethyl)oxetan-3-yl)methoxy)phenyl)nicotinamide

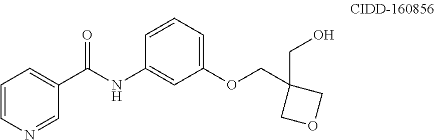 [SMILES: O=C(Nc1cccc(OCC2(CO)COC2)c1)c1cccnc1]

¹H NMR (400 MHz, DMSO) δ 10.37 (s, 1H), 9.07 (d, J=1.7 Hz, 1H), 8.73 (dd, J=4.8, 1.5 Hz, 1H), 8.29-8.23 (m, 1H), 7.54 (dd, J=7.7, 5.1 Hz, 1H), 7.51 (t, J=1.9 Hz, 1H), 7.32 (d, J=8.6 Hz, 1H), 7.25 (t, J=8.1 Hz, 1H), 6.73 (dd, J=8.1, 1.7 Hz, 1H), 4.98 (t, J=4.4 Hz, 1H), 4.39 (dd, J=15.0, 5.9 Hz, 4H), 4.10 (s, 2H), 3.69 (d, J=4.1 Hz, 2H). ESI-MS m/z [M+H]⁺: 315.2

1. A compound of Formula I: 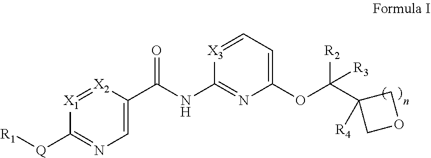
wherein,
R1 is hydrogen; C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; or C₃, C₄, C₅, C₆, or C₇ cycloalkyl; and is optionally substituted;
R2 is hydrogen; C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; or C₃, C₄, C₅, C₆, or C₇ cycloalkyl; and is optionally substituted;
R3 is hydrogen; C₁, C₂, C₃, C₄, C₅, or C₆ saturated alkyl; C₁, C₂, C₃, C₄, C₅, or C₆ mono-unsaturated alkyl; C₁, C₂, C₃, C₄, C₅, or C₆ poly-unsaturated alkyl; or C₃, C₄, C₅, C₆, or C₇ cycloalkyl; and is optionally substituted;
Q is oxygen or a sigma bond;
X₁ is CH, nitrogen (N), or a carbon substituted with a halogen, a nitrogen, or an R₁ group as defined above;
X₂ is CH, nitrogen (N), or a carbon substituted with a halogen, a nitrogen, or an R₁ group as defined above;
X₃ is CH, nitrogen (N), or a carbon substituted with a halogen, a nitrogen, or an R₁ group as defined above;
X₄ is CH, nitrogen (N), or a carbon substituted with a halogen, a nitrogen, or an R₁ group as defined above;
n is 1, 2, or 3.

2. The compound of claim 1, wherein R1 is a methyl or substituted methyl.

3. The compound of claim 1, wherein R1 is substituted with 1, 2, or 3 halogens; C1, C2, or C3 alkyl; C₄, C₅, C₆, or C₇ cycloalkyl; heterocycle; aryl; or heteroaryl.

4. The compound of claim 1, wherein R2 is a methyl or substituted methyl.

5. The compound of claim 1, wherein R2 is substituted with 1, 2, or 3 halogens; C1, C2, or C3 alkyl; C₄, C₅, C₆, or C₇ cycloalkyl; heterocycle; aryl; or heteroaryl.

6. The compound of claim 1, wherein R3 is a methyl or substituted methyl.

7. The compound of claim 1, wherein R3 is substituted with 1, 2, or 3 halogens; C1, C2, or C3 alkyl; C₄, C₅, C₆, or C₇ cycloalkyl; heterocycle; aryl; or heteroaryl.

8. The compound of any one of claims 1 to 7, wherein Q is oxygen and R₁ is methyl.

9. The compound of any one of claims 1 to 7, wherein Q is a sigma bond and R1 is hydrogen.

10. The compound of any one of claims 1 to 9, wherein R2 is hydrogen.

11. The compound of any one of claims 1 to 10, wherein R3 is hydrogen.

12. The compound of any one of claims 1 to 11, wherein X1 is carbon.

13. The compound of any one of claims 1 to 12, wherein X2 is carbon.

14. The compound of any one of claims 1 to 13, wherein X3 is carbon.

15. The compound of any one of claims 1 to 14, wherein X4 is carbon.

16. The compound of any one of claims 1 to 15, wherein X1 is nitrogen.

17. The compound of any one of claims 1 to 16, wherein X2 is nitrogen.

18. The compound of any one of claims 1 to 17, wherein X3 is nitrogen.

19. The compound of any one of claims 1 to 18, wherein X4 is nitrogen.

20. The compound of any one of claims 1 to 19, wherein R4 is hydrogen, methyl, ethyl, hydroxyl or methyl ester.

21. The compound of claim 1, wherein the compound is 6-methoxy-N-(3-(oxetan-3-ylmethoxy)phenyl)nicotinamide, 4-methoxy-N-(3-(oxetan-3-ylmethoxy)phenyl)benzamide, 4-methoxy-N-(3-((3-methyloxetan-3-yl)methoxy)phenyl)benzamide, 4-methoxy-N-(3-((3-ethyloxetan-3-yl)methoxy)phenyl)benzamide, 4-methoxy-N-(3-((3-ethyloxetan-3-yl)methoxy)phenyl)benzamide, 6-methoxy-N-(4-(oxetan-3-ylmethoxy)pyridin-2-yl)nicotinamide, 6-methoxy-N-(4-((3-methyloxetan-3-yl)methoxy)pyridin-2-yl)nicotinamide, 6-methoxy-N-(4-((3-ethyloxetan-3-yl)methoxy)pyridin-2-yl)nicotinamide, N-(4-((3-(hydroxymethyl)oxetan-3-yl)methoxy)pyridin-2-yl)-6-methoxynicotinamide, N-(3-(oxetan-3-ylmethoxy)phenyl)nicotinamide, N-(3-((3-methyloxetan-3-yl)methoxy)phenyl)nicotinamide, N-(3-((3-ethyloxetan-3-yl)methoxy)phenyl)benzamide, or N-(3-((3-(hydroxymethyl)oxetan-3-yl)methoxy)phenyl)nicotinamide.

22. A method of treating fungal infections comprising administering one or more compounds of claim 1 to 16 to a subject at risk of acquiring or having a fungal infection.

23. The method of claim 22, wherein the fungal infection is a Candida infection.

24. The method of claim 23, wherein the Candida infection is a Candida dubliniensis, Candida parapsilosis, Candida tropicalis, Candida kerfyr, Candida guilliermondii, Candida inconspicua, Candida famata, Candida glabrata, Candida krusei, or Candida lusitaniae infection.

25. The method of claim 22, wherein the fungal infection is a Candida albicans infection.

26. The method of claim 22, wherein the subject is hospitalized.

27. The method of claim 22, wherein the subject is in a facilitated living facility.

28. The method of claim 22, wherein the subject is immune-compromised.

29. The method of claim 22, wherein the subject is intubated, catheterized, or harbors a medical implant.
